(19) United States
(12) Patent Application Publication
(10) Pub. No.: US 2025/0374963 A1
(43) Pub. Date: Dec. 11, 2025

(54) ELECTRONIC VAPING DEVICE WITH LOW-PROFILE FLANGES

(71) Applicant: Altria Client Services LLC, Richmond, VA (US)

(72) Inventors: Joshua J. NELSON, Richmond, VA (US); Jose R. BONET, Richmond, VA (US); Sean C. KENNEDY, Richmond, VA (US)

(73) Assignee: Altria Client Services LLC, Richmond, VA (US)

(21) Appl. No.: 19/228,090

(22) Filed: Jun. 4, 2025

Related U.S. Application Data
(60) Provisional application No. 63/656,717, filed on Jun. 6, 2024.

(51) Int. Cl. A24F 40/40; A24F 40/10; A61M 11/04

(52) U.S. Cl. CPC A24F 40/40; A24F 40/10; A61M 11/042

(57) ABSTRACT

A receiver assembly of an e-vaping device includes an enclosed portion and a receptacle portion. The enclosed portion has an upper surface, and the receptacle portion is configured to receive a cartridge bottom of a removable vaping cartridge. The receptacle portion includes a receptacle bottom defined by the upper surface of the enclosed portion, front and back flanges having a flange height measured from the receptacle bottom, sidewalls having a sidewall height measured from the receptacle bottom, and an upper receptacle boundary defined by the sidewall height. The sidewall height is short enough such that at least a portion of a heating chamber included in a removable vaping cartridge remains above the sidewalls while the cartridge bottom of the removable vaping cartridge is inserted in the receptacle portion.

### CROSS-REFERENCE TO RELATED APPLICATION

This application claims benefit under 35 U.S.C. 119(e) of U.S. Provisional Application No. 63/656,717 entitled, “Electronic Vaping Device With Low-Profile Flanges,” filed on Jun. 6, 2024, which is incorporated herein by reference in its entirety for all purposes.

### BACKGROUND

### Field

The present disclosure relates to electronic vaping devices and cartridges, and more particularly to electronic vaping devices with low-profile flanges.

### Description of Related Art

An electronic vaping (“e-vaping”) system generally includes an on-board power source, such as a battery, which is electrically connected to a heating element that converts a pre-vapor formulation stored in a cartridge to a vapor. The vapor exits the e-vaping system through a mouthpiece including at least one outlet. E-vaping systems are generally manufactured as either inseparable systems or separable systems. An inseparable system is a unitary assembly that is intended to remain intact after being manufactured, while separable systems are manufactured in at least two separate parts which may be assembled at some point after being manufactured. Another characteristic of inseparable system is that the unitary assembly houses both the on-board power source and the pre-vapor formulation. Separable systems generally include a body, sometimes referred to as a “battery section” or “device” that houses the on-board power source, and a removable cartridge or pod that houses the pre-vapor formulation.

### SUMMARY

As used herein, unless otherwise required by context or explicitly noted, the term “e-vaping system” is used to refer to a complete e-vaping device, while the term “e-vaping device” or “receiver assembly” are used to refer to a portion of an e-vaping system that houses an on-board power source, such as a battery, and which a removable vaping cartridge may be mated, docked, inserted, or otherwise attached.

Furthermore, unless otherwise required by context or explicitly noted, the terms “cartridge,” “pod,” “removable pod or cartridge,” “removable vaping pod or cartridge,” and similar terms are used to refer to the portion of a separable e-vaping system that stores the pre-vapor formulation and may be mated, docked, inserted, or attached to the receiver assembly of the separable e-vaping system.

Some example embodiments of e-vaping devices disclosed herein include an e-vaping device with a receiver assembly including both a closed portion that may house electrical/electronic elements, and a receptacle portion having an open top and configured to receive a cartridge bottom of a removable vaping cartridge. The bottom of the receptacle portion may be coextensive with an upper surface of the closed portion of the receiver assembly. Two opposing sides of the receptacle portion may include sidewalls that define the upper boundary of the receptacle portion. Other sides of the receptacle portion may include flanges that assist the sidewalls in maintaining a removable vaping cartridge in a docked/mated/inserted position. Using low-profile sidewalls and/or flanges allows some or all vapor generating elements of a removable vaping cartridge to remain above/outside the receptacle portion of the receiver assembly during a time the removable vaping cartridge is mated/docked with the receiver assembly.

In some example embodiments, positioning some or all of the vapor generating elements above/outside the receptacle portion while the removable vaping cartridge is mated to the receiver assembly may limit the potential for device overheating. Furthermore, in some implementations, the use of low-profile sidewalls and/or flanges may reduce the amount of material used in constructing the receiver assembly. In further example embodiments, low-profile sidewalls and/or flanges may provide additional manufacturing freedoms related to placement of vapor producing elements within a heating chamber, and/or provide additional manufacturing freedoms with regard to a size or location of the heating chamber.

In at least one example embodiment, a receiver assembly of an e-vaping device includes an enclosed portion having an upper surface; and a receptacle portion configured to receive a cartridge bottom of a removable vaping cartridge. The receptacle portion includes a receptacle bottom defined by the upper surface of the enclosed portion, front and back flanges having a flange height measured from the receptacle bottom, sidewalls having a sidewall height measured from the receptacle bottom, and an upper receptacle boundary defined by the sidewall height, the sidewall height being short enough such that at least a portion of a heating chamber included in a removable vaping cartridge remains above the sidewalls while the cartridge bottom of the removable vaping cartridge is inserted in the receptacle portion.

In certain example embodiments, a receiver assembly of an e-vaping device includes an enclosed portion having an upper surface; and a receptacle portion configured to receive a cartridge bottom of a removable vaping cartridge. The receptacle portion includes a receptacle bottom defined by the upper surface of the enclosed portion, front and back flanges having a flange height measured from the receptacle bottom, sidewalls having a sidewall height measured from the receptacle bottom, and an upper receptacle boundary defined by the sidewall height, the sidewall height being short enough such that a heater included in a removable vaping cartridge remains above the sidewalls while the cartridge bottom of the cartridge bottom of the removable vaping cartridge is inserted in the receptacle portion.

In various example embodiments, a receiver assembly of an e-vaping device includes an enclosed portion having an upper surface; and a receptacle portion configured to receive a cartridge bottom of a removable vaping cartridge, the receptacle portion including a receptacle bottom defined by the upper surface of the enclosed portion, front and back flanges having a flange height measured from the receptacle bottom, sidewalls having a sidewall height measured from the receptacle bottom, and an upper receptacle boundary defined by the sidewall height, the sidewall height being short enough such that a fluid chamber included in a removable vaping cartridge remains above the sidewalls while the cartridge bottom of the cartridge bottom of the removable vaping cartridge is inserted in the receptacle portion.

At least one further example embodiment includes an e-vaping device, comprising a receiver assembly; and a removable vaping cartridge mated to the receiver assembly, the removable vaping cartridge including a cartridge bottom, and a heating chamber positioned within the removable vaping cartridge, the receiver assembly including an enclosed portion having an upper surface, a receptacle portion configured to receive a cartridge bottom of the removable vaping cartridge, the receptacle portion including a receptacle bottom defined by the upper surface of the enclosed portion, front and back flanges having a flange height measured from the receptacle bottom, sidewalls having a sidewall height measured from the receptacle bottom, and an upper receptacle boundary defined by the sidewall height, the sidewall height being short enough such that at least a portion of the heating chamber included in the removable vaping cartridge remains above the sidewalls while the cartridge bottom of the removable vaping cartridge is inserted in the receptacle portion of the receiver assembly.

Yet another example embodiment includes an e-vaping device, comprising a receiver assembly; and a removable vaping cartridge mated to the receiver assembly, the removable vaping cartridge including a cartridge bottom, and a heater positioned within the removable vaping cartridge, the receiver assembly including an enclosed portion having an upper surface, a receptacle portion configured to receive a cartridge bottom of the removable vaping cartridge, the receptacle portion including a receptacle bottom defined by the upper surface of the enclosed portion, front and back flanges having a flange height measured from the receptacle bottom, sidewalls having a sidewall height measured from the receptacle bottom, and an upper receptacle boundary defined by the sidewall height, the sidewall height being short enough such that the heater included in the removable vaping cartridge remains above the sidewalls while the cartridge bottom of the removable vaping cartridge is inserted in the receptacle portion of the receiver assembly.

A further example embodiment includes an e-vaping device, comprising a receiver assembly; and a removable vaping cartridge mated to the receiver assembly, the removable vaping cartridge including a cartridge bottom, and a fluid chamber within the removable vaping cartridge, the fluid chamber configured to hold a pre-vapor solution, the receiver assembly including an enclosed portion having an upper surface, a receptacle portion configured to receive a cartridge bottom of the removable vaping cartridge, the receptacle portion including a receptacle bottom defined by the upper surface of the enclosed portion, front and back flanges having a flange height measured from the receptacle bottom, sidewalls having a sidewall height measured from the receptacle bottom, and an upper receptacle boundary defined by the sidewall height, the sidewall height being short enough such that the fluid chamber included in the removable vaping cartridge remains above the sidewalls while the cartridge bottom of the removable vaping cartridge is inserted in the receptacle portion of the receiver assembly.

### BRIEF DESCRIPTION OF THE DRAWINGS

The various features and advantages of the non-limiting embodiments herein may become more apparent upon review of the detailed description in conjunction with the accompanying drawings. The accompanying drawings are provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1A is a side view of a fully assembled, separable e-vaping system in accordance with some example embodiments;

FIG. 1B is a top view of the fully assembled, separable e-vaping system illustrated in FIG. 1A, in accordance with some example embodiments;

FIG. 2 is a cutaway view of a fully assembled, separable e-vaping system, in accordance with some example embodiments;

FIG. 3A is a perspective view of a fully assembled, separable e-vaping system, in accordance with some example embodiments;

FIGS. 3B and 3C are end views of a fully assembled, separable e-vaping system, in accordance with some example embodiments;

FIG. 4A is a perspective view of a receiver assembly of a separable e-vaping system, in accordance with some example embodiments;

FIG. 4B is an end view of an upper surface of an enclosed portion of a receiver assembly of a separable e-vaping system, in accordance with some example embodiments;

FIG. 5A is side view of a receiver assembly of a separable e-vaping system, in accordance with some example embodiments;

FIG. 5B is a cross section of the receiver assembly shown in FIG. 5A, in accordance with some example embodiments;

FIG. 5C is a block diagram illustrating a power control system of a separable e-vaping system, in accordance with some example embodiments;

FIG. 6A is a perspective view of a removable vaping cartridge, in accordance with some example embodiments;

FIG. 6B is a bottom view of a removable vaping cartridge cover, in accordance with some example embodiments;

FIG. 7A is side view of a removable vaping cartridge, in accordance with some example embodiments;

FIG. 7B is a cross section of a removable vaping cartridge, in accordance with some example embodiments; and

FIG. 8 is a perspective view of a heater included in a removable vaping cartridge, in accordance with various example embodiments.

### DETAILED DESCRIPTION OF EXAMPLE EMBODIMENTS

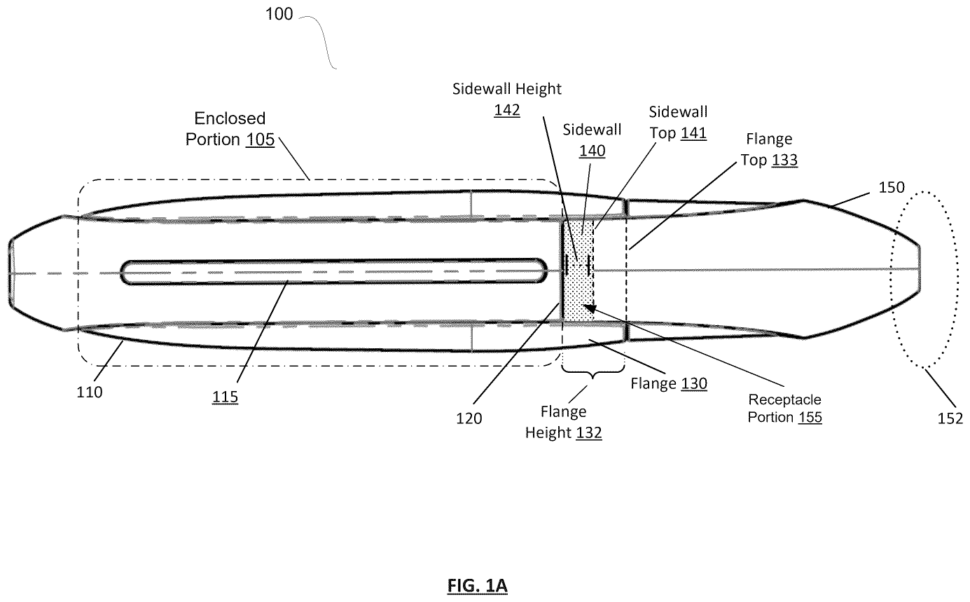

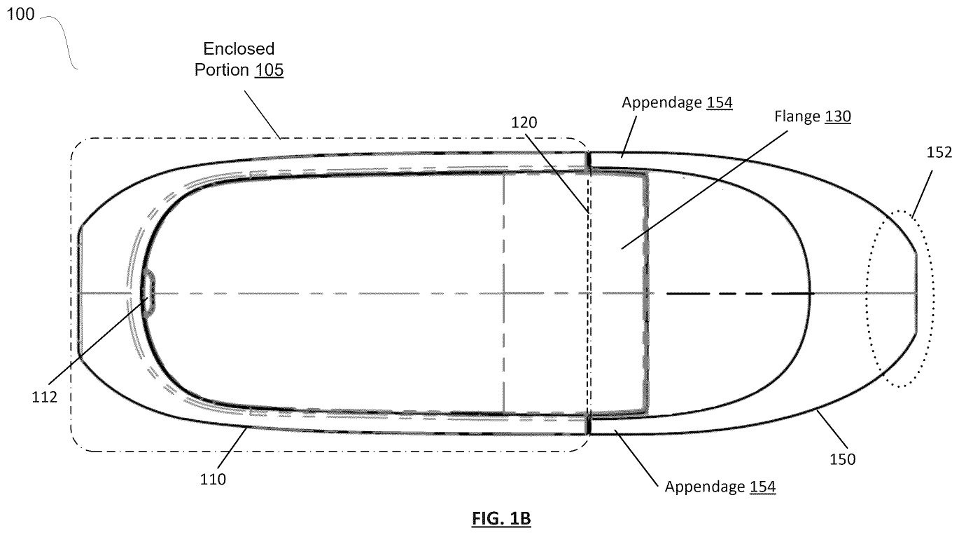

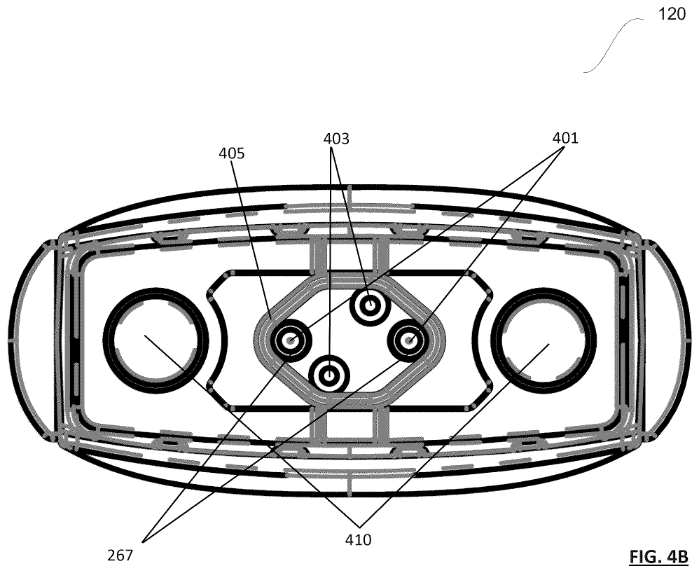

Referring first to FIGS. 1A and 1B, a fully assembled, separable e-vaping system 100 will be discussed in accordance with some example embodiments. As shown, separable e-vaping system 100 includes receiver assembly or device 110 and removable vaping pod or cartridge 150 docked/mated with receiver assembly or device 110. Receiver assembly 110 includes enclosed portion 105 and receptacle portion 155. With reference to FIG. 4A, showing a right, perspective view of the receiver assembly or device 110, an upper surface 120 of enclosed portion 105 defines a bottom of receptacle portion 155, and the upper boundary of receptacle portion 155 corresponds to sidewall top 141 of sidewalls 140 located on two opposing sides of receptacle portion 155. Flanges 130 are located on two other opposing sides of receptacle portion 155, providing both front flange 130b and back flange 130a. Sidewalls 140 have a sidewall height 142, as measured from upper surface 120 of enclosed portion 105 to sidewall top 141. Flanges 130 have a flange height 132, measured from upper surface 120 of enclosed portion 105 to flange top(s) 133. Receiver assembly 110 may optionally include decoration/grip 115, and visual indicator 112.

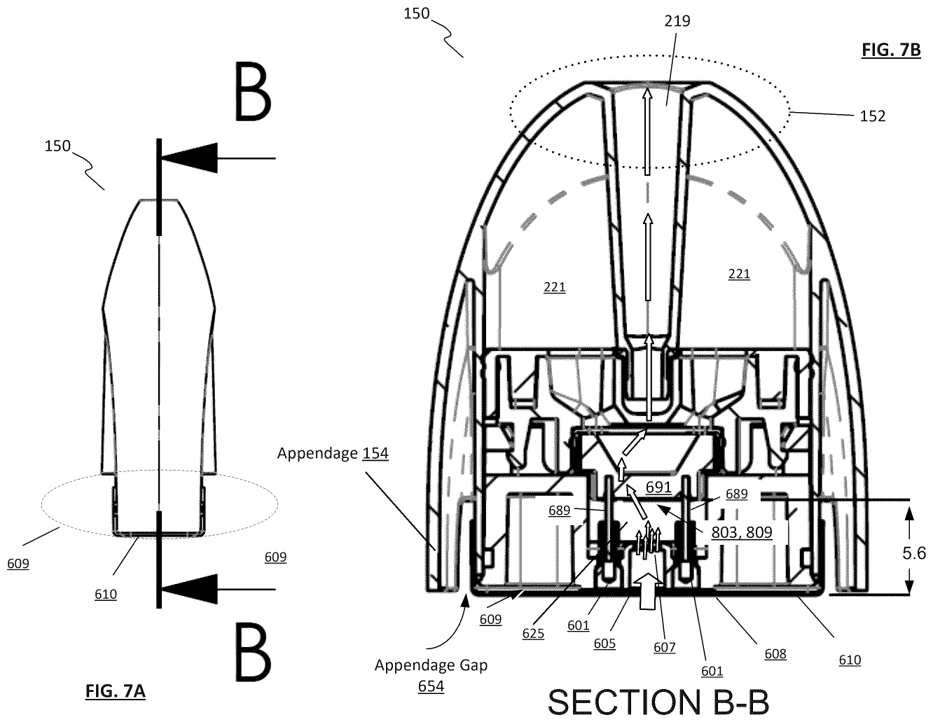

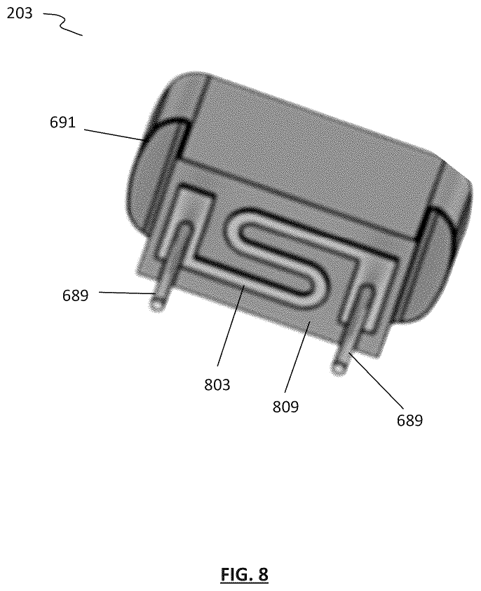

Returning to FIGS. 1A, 1B and FIGS. 7A, 7B, removable vaping cartridge 150 includes mouth end 152, and a cartridge bottom 609. As used herein, a “mouth end” is to be understood as distinct from a “mouthpiece.” In particular, unless otherwise explicitly defined or required by context, the term mouth end is used herein to refer to an integrated termination of a housing, cover, or the like, while the term mouthpiece is an individually distinguishable item that is part of an e-vaping device.

In at least one example embodiment, cartridge bottom 609 is configured to slide into receptacle portion 155 of receiver assembly or device 110 until stopped by upper surface 120 of receiver assembly or device 110. As illustrated in FIGS. 1B and 7B, removable vaping cartridge 150 may include appendages 154, which are spaced apart from cartridge bottom 609 and configured to permit sidewalls 140 (not visible in these views) to slide between appendages 154 and cartridge bottom 609.

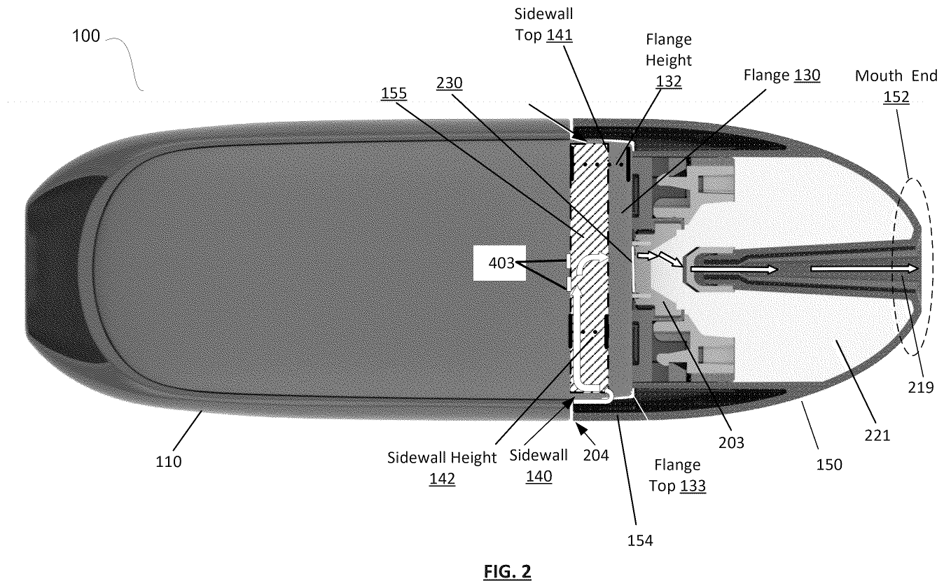

Referring next to FIG. 2, a cutaway view of a fully assembled, separable e-vaping system 100 will be discussed in accordance with various example embodiments. Various internal elements of removable vaping cartridge 150, including vapor producing elements, along with an airpath for delivering an inhalable dispersion through mouth end 152, are illustrated and discussed.

In the example embodiment illustrated in FIG. 2, removable vaping cartridge 150 includes a mouth end 152 incorporating a vapor delivery channel 219, through which an inhalable dispersion may be drawn. The term “inhalable dispersion” as used herein includes reference to a volume of air including an entrained vapor. In the following discussion the white arrows illustrate airflow according to an example embodiment.

To generate the inhalable dispersion, in response to a draw being initiated through vapor delivery channel 219, air may be drawn into separable e-vaping system 100 though air gap 204, until it meets sidewall 140, at which point the air is routed over sidewall top 141, into receptacle portion 155 of receiver assembly or device 110, and up into removable vaping cartridge 150. Drawing air from receptacle portion 155 into removable vaping cartridge communicates a negative draw pressure, or simply negative pressure, through the negative pressure ports 403 into enclosed portion 105 of receiver assembly or device 110.

Inside removable vaping cartridge 150 the air is drawn around heater 203, where a vapor is entrained in the air flowing past heater 203, into vapor delivery channel 219, and out mouth end 152. Note that receptacle portion 155 is shown with hatching to illustrate its location, but in fact would not be physically visible in this view.

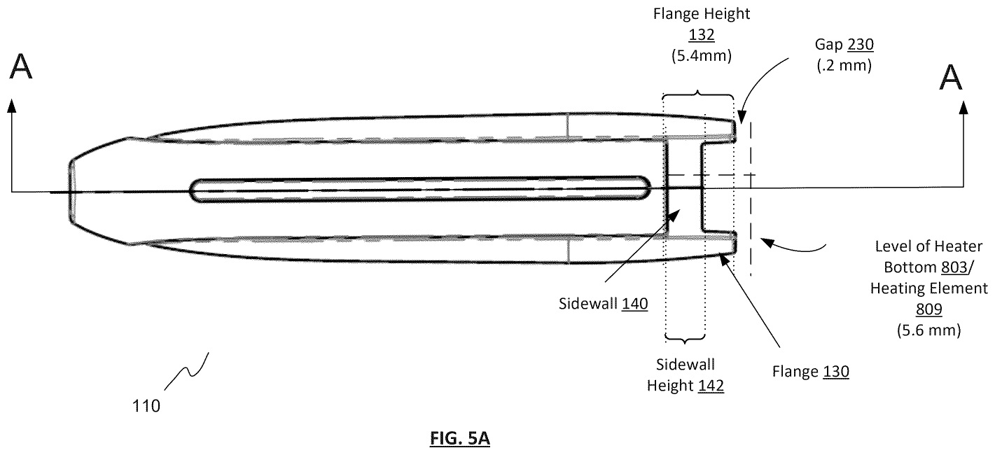

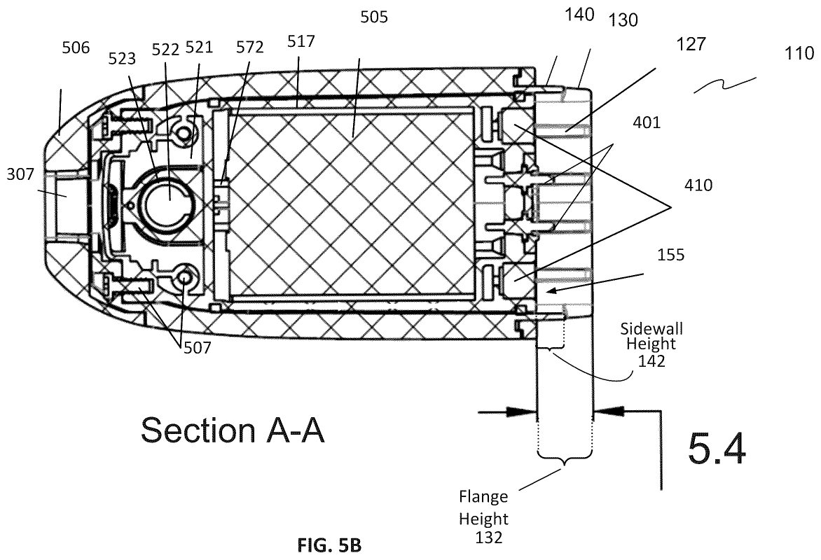

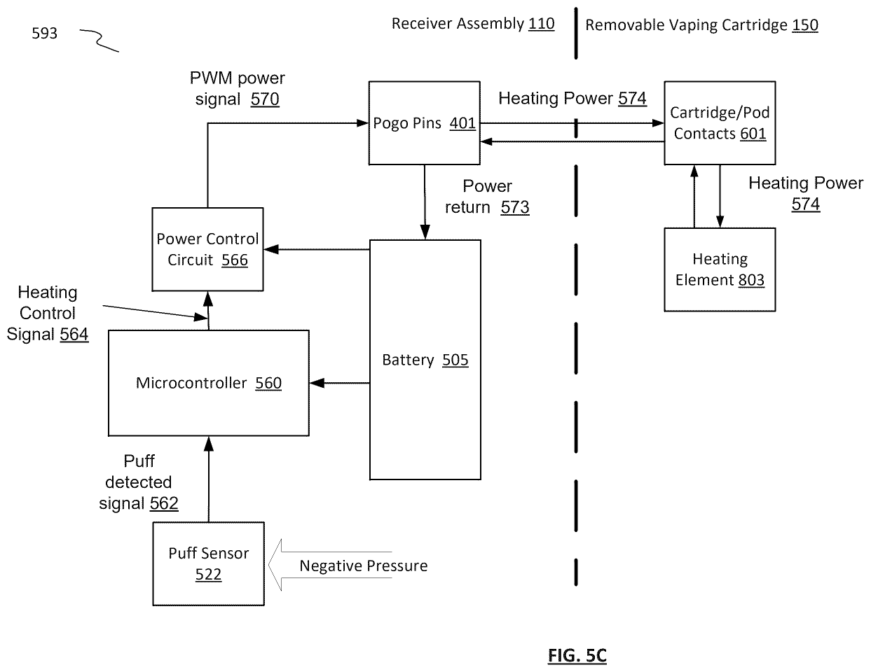

The process by which the vapor is entrained in the air may include the following: a pre-vapor solution stored in a fluid chamber 221 is absorbed into a porous ceramic body of heater 203. A heating element in contact with a bottom surface of the porous ceramic body heats the absorbed pre-vapor solution to form the vapor, which is entrained in the passing air. In general, the receiver assembly or device 110 supplies power to the heater in response to an activation signal generated by a puff sensor (illustrated in FIG. 5B). The activation signal is generated in response to the puff sensor sensing the negative pressure communicated via negative pressure ports 403 by a draw on mouth end 152. Various examples of heater types, heater and e-vaping control mechanisms, puff sensors, air seals, airflow paths, and the like can be found in U.S. patent application Ser. No. 18/588,408, filed Feb. 27, 2024 and entitled “Electronic Vaping Devices, Cartridges, And Access Control Systems,” the entire contents of which is incorporated herein by reference.

It will be noted with reference to FIG. 2, that there is a gap 230 between the top of the flange top(s) 133 and the bottom of heater 203. Thus, in the illustrated example embodiment, heater 203, fluid chamber 221, and at least a portion of the heating chamber, or heater chamber, (illustrated further in FIG. 7B) all remain outside of receptacle portion 155, the upper boundary of which is defined by the sidewall height. Further note that in the illustrated example embodiment, sidewall height 142 and flange height 132 are both short enough so that heater 203, reservoir or fluid chamber 221, and at least a portion of the heating chamber remain above both the sidewalls and front and back flanges 130.

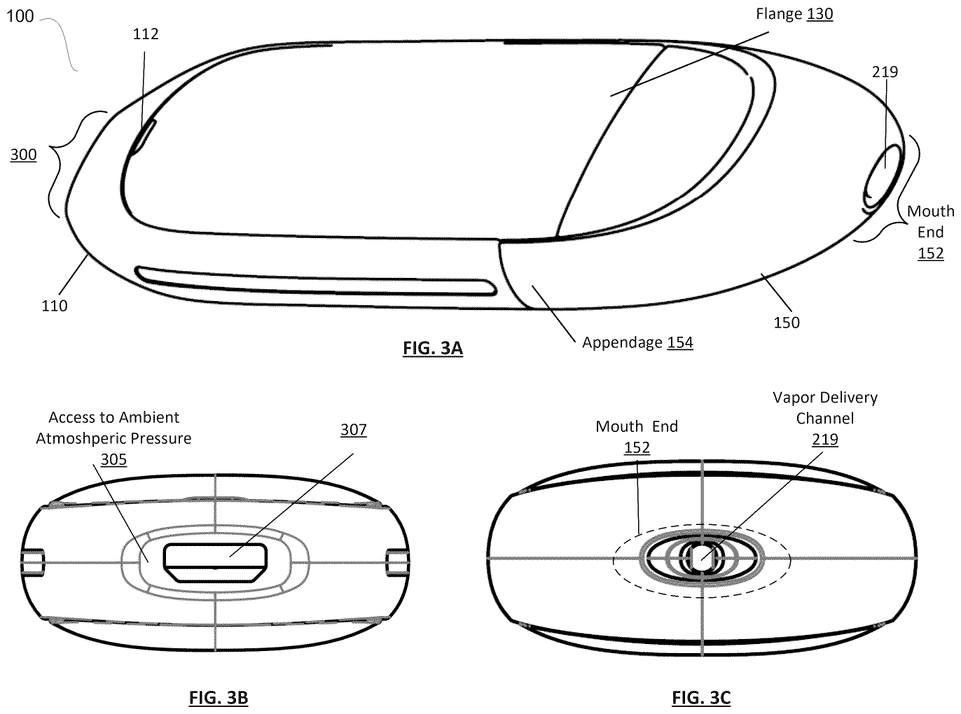

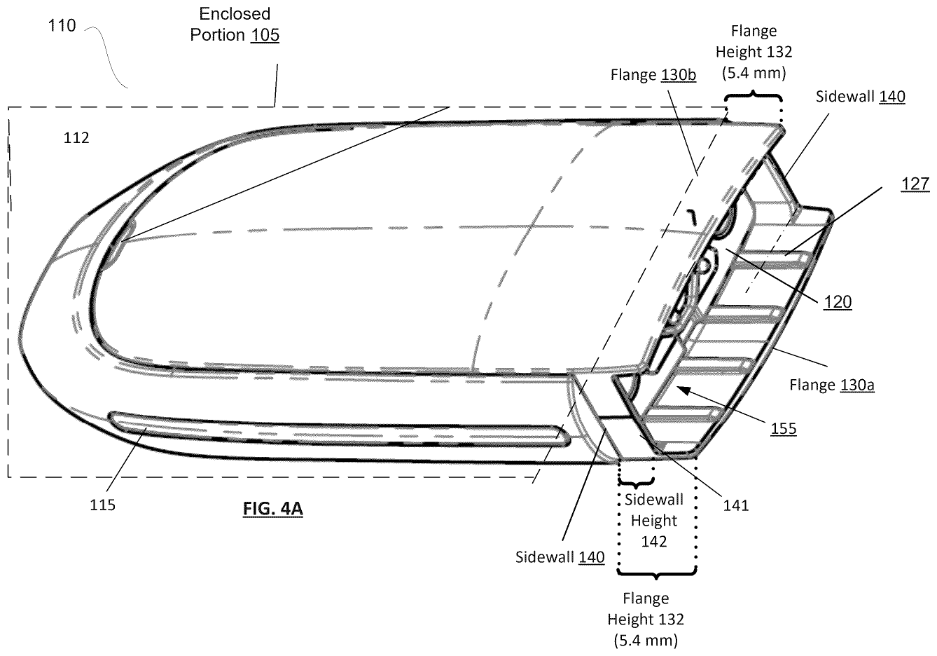

Referring next to FIGS. 3A-3C a fully assembled, separable e-vaping system 100 will be discussed in accordance with some example embodiments. Separable e-vaping system 100 includes receiver assembly or device 110 and removable vaping cartridge 150. In addition to the elements and features previously discussed, receiver assembly or device 110 also includes a charging port 307 at bottom end 300, which may provide access to ambient atmospheric pressure 305. Charging port 307 may include a universal serial bus (USB) port, or some other suitable charging and/or communications port.

A top of removable vaping cartridge 150, as illustrated in FIG. 3C, shows a top view of mouth end 152 and vapor delivery channel 219.

Referring next to FIG. 4A, a receiver assembly or device 110 will be discussed in accordance with some example embodiments. In the illustrated example embodiment, receiver assembly or device 110 include front flange 130b, back flange 130a, and upper surface 120. Front flange 130b and back flange 130a are provided with ribs 127 integral to front flange 130b and back flange 130a. In operation, ribs 127 may be used to provide a tighter friction fit with an inserted removable vaping cartridge. In some example embodiments, corresponding ribs may be formed on the removable vaping cartridge, or on a cartridge bottom cover.

Referring next to FIG. 4B, an upper surface 120 of an enclosed portion 105 of a receiver assembly or device 110 of a separable e-vaping system 100 will be discussed in accordance with various example embodiments. Upper surface 120 may be formed of a rigid material, such as polyethylene, having grooves and openings formed therein to accommodate various elements. As noted earlier, upper surface 120 also defines the bottom of receptacle portion 155.

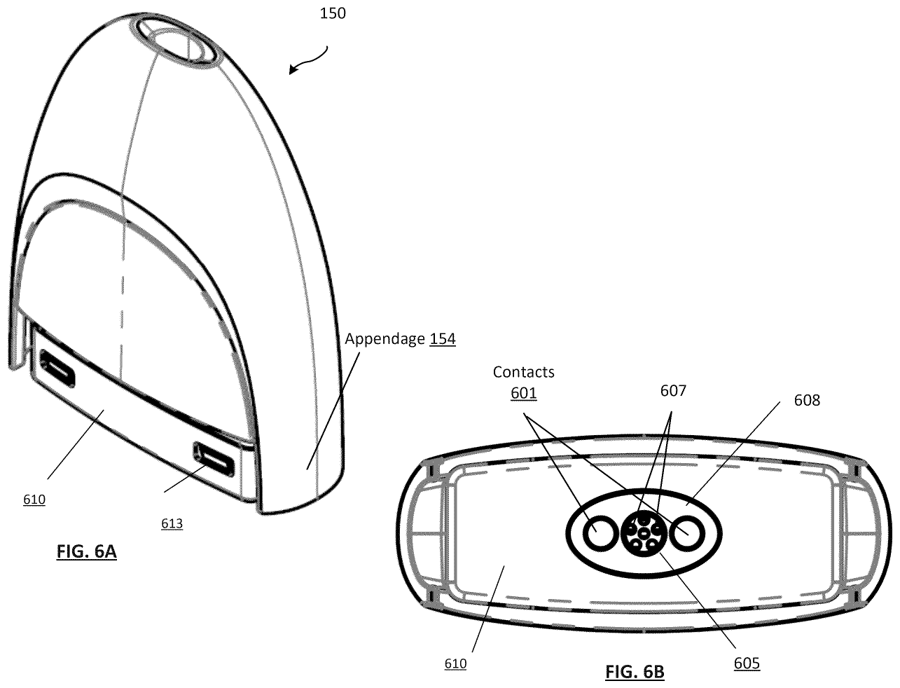

As will be discussed subsequently with respect to FIGS. 6B and 7B, there is at least one opening in the bottom portion of the removable vaping cartridge 150 that provides fluid communication between receptacle portion 155 and a heating chamber included in removable vaping cartridge 150.

Upper surface 120 of enclosed portion 105 includes openings to receive magnets 410, which work in conjunction with a metallic cartridge bottom cover to keep a removable vaping cartridge 150 in place. Also included in upper surface 120 are openings 267 to receive pogo pins 401 or other power connectors used to provide power to a heating element of a heater included in removable vaping cartridge 150. Negative pressure ports 403 allow changes in pressure within receptacle portion 155 to be sensed by a puff sensor within the enclosed portion 105 of receiver assembly or device 110. In at least one example embodiment, an airflow sensor/puff sensor may include an air-flow sensor as described in U.S. Pat. No. 9,072,321 issued on Jul. 7, 2015, the entire contents of which is incorporated herein by reference.

The upper surface 120 of enclosed portion 105 also includes seal 405, which helps prevent an fluid that may reach the upper surface 120 from entering receiver assembly or device 110.

Referring next to FIG. 5A a side view of a receiver assembly of a separable e-vaping system will be discussed in accordance with some example embodiments. In an example embodiment, a flange height 132 of both flanges 130 may be about 5.4 mm or less, and a sidewall height 142 of both sidewalls 140 may be even shorter than the flange height 132. In at least one example embodiment discussed herein subsequently with reference to FIG. 7B, a lower surface 809 of heater 203 may be about 5.6 mm above a bottom cover 610 covering the cartridge bottom 609. In embodiments employing flanges 130 having a flange height of 6.17 mm., a gap of 0.2 mm exists between the top of the flanges 130 and the lower surface 809 of the porous ceramic body 691, and a slightly smaller, gap exists between the flanges 130 and the heating element 803.

Referring next to FIG. 5B, FIG. 5B is a cross section of the receiver assembly or device 110, which includes a puff sensor seal 523 configured to form a seal about a puff sensor 522. Puff sensor seal 523 isolates a sensing portion of puff sensor 522 from ambient air pressure. In at least one example embodiment, the puff sensor 522 may include a sensor as described in U.S. Pat. No. 9,072,321 issued on Jul. 7, 2015, the entire contents of which is incorporated herein by reference.

Some example embodiments of a receiver assembly or device 110 also include printed circuit board assembly (PCBA) 521; screws 507 fastening PCBA 521 to a housing 506; a battery holder 517 mounted within housing 506, and configured to hold a battery 505 having battery contacts 572; and magnets 410 inserted into a bottom surface of the receptacle portion 155, which corresponds to an upper surface of the housing 506. The magnets 410 are configured to magnetically engage a docked removable vaping cartridge (not shown in this view).

The illustrated example embodiment of receiver assembly or device 110 also includes device electrical contacts, which may include pogo pins 401 extending beyond an upper surface 120 of the receptacle portion 155 of the housing 506. The pogo pins 401 provide a springing action, allowing the device contact (e.g. pogo pins 401) to firmly contact corresponding cartridge/pod contacts 601 (see FIG. 6B) included in a removable vaping cartridge. The pogo pins 401 serve to route battery power to a docked, removable vaping cartridge under control of a microcontroller, processor, or other circuitry mounted on the back side of PCBA 521. In at least one example embodiment, wires run behind battery 505 and connect the pogo pins 401 to the PCBA 521 and/or the battery 505, thereby supplying power.

Referring next to FIG. 5C, a block diagram illustrating a power control system 593 of a separable e-vaping system 100 will be discussed in accordance with some example embodiments. The power control system 593 includes the puff sensor 522, a microcontroller 560, the battery 505, a power control circuit 566, and the pogo pins 401, each of which is part of the receiver assembly 110. The battery 505, puff sensor 522, and pogo pins 401 are illustrated in FIG. 5B. Referring briefly to FIG. 7B, the pogo pins 401 contact the cartridge/pod contacts 601 when removable vaping cartridge 150 is received in the receptacle portion 155. The cartridge pod contacts 601 extend into the removable vaping cartridge 150 and are connected to either end of a heating element 803 (see also FIG. 8). The microcontroller 560 and the power control circuit 566 may be mounted on a side of the PCBA 521 opposite the side of the PCBA 521 on which the puff sensor 522 is mounted, and are hidden from view in FIG. 5B. The elements of the power control system 593 may be physically connected using traces included on the PCBA 521, or wires (not illustrated).

An example of operation of the separable e-vaping system 100 will now be discussed with reference to FIGS. 2 and 5C. With reference first to FIG. 2, an example of operation, a draw on a mouth end 152 of a removable vaping cartridge 150 causes negative pressure to be communicated to the puff sensor 522 via negative pressure ports 403.

With reference next to FIG. 5C, the negative pressure is sensed by the puff sensor 522, which transmits a puff detected signal 562 to the microcontroller 560. The puff detected signal 562 indicates to microcontroller 560 that a draw is occurring, and that power should be supplied to heating element 803. In at least one example embodiment, puff sensor 522 senses the negative pressure created by the draw based on a pressure differential between ambient pressure present on one side of puff sensor 522 and the negative pressure applied to the sensing side of the puff sensor 522.

In response to receiving the puff detected signal 562, the microcontroller 560 transmits a heating control signal 564 to power control circuit 566, which in at least one example embodiment may include a power transistor or the like, and may generate a pulse width modulated (PWM) power signal 570. Note that in some example embodiments the power control circuit 566 may simply provide a path to either the positive or negative terminal of battery 505. The Power control circuit 566 transmits the (PWM) power signal 570 to pogo pins 401. In at least one example embodiment, one of two pogo pins 401 is connected to battery 505 to complete a power return 573 side of an electrical circuit, while a second of the two pogo pins 401 is connected to power control circuit 566. In some example embodiments, both pogo pins 401 are controllably connected to battery 505 through power control circuit 566.

The heating power 574 generated by receiver assembly 110 is supplied to the cartridge/pod contacts 601 included in removable vaping cartridge 150 via pogo pins 401. The cartridge/pod contacts 601 are connected to contact posts 689, which are in turn connected to the heating element 803. As such the heating power 574 is supplied to the heating element 803 via the cartridge pod contacts 601 and contact posts 689.

Referring again briefly to FIG. 2, in response to receiving heating power 574, heating element 803 heats a pre-vapor formulation to generate an inhalable dispersion, which is entrained in air flowing past heater 203, and delivered out vapor delivery channel 219.

Referring next to FIGS. 6A and 6B a bottom cover 610 of a removable vaping cartridge 150 will be discussed in accordance with various example embodiments. As illustrated by FIG. 6A, appendages 154 extend generally to the bottom of bottom cover 610. The bottom cover 610 may be snapped to the bottom portion of removable vaping cartridge 150 using elastic protrusions 613 formed on the bottom portion of removable vaping cartridge 150.

As shown by the bottom view of bottom cover 610 provided in FIG. 6B, the bottom cover 610 includes oval cut-out 608 providing an opening to expose electrical cartridge/pod contacts 601 and an airflow opening 605. Airflow opening 605 allows airflow from receptacle portion 155 of receiver assembly or device 110 into a heating chamber (not visible in this view) included in removable vaping cartridge 150. The airflow opening 605 leads to five airflow passages 607 (see also FIG. 7B) shown as open circles within airflow opening 605. While five airflow passages have been shown, there may be a fewer or greater number of airflow passages. As further shown in FIG. 7B, airflow through the airflow passages 607 lead to the heater on the bottom of the porous ceramic body.

Note that in at least one example embodiment, bottom cover 610 includes metal to cooperate with magnets 410 included in the receiver assembly or device 110 to hold bottom cover 610 in place.

Referring next to FIGS. 7A and 7B, some example embodiments of a removable vaping cartridge 150 will be discussed. As illustrated by FIG. 7A, removable vaping cartridge 150 includes a cartridge bottom 609 and bottom cover 610 covering cartridge bottom 609. The bottom cover 610 includes metal for making a magnetic connections to magnets 410 exposed in the receptacle portion (see FIG. 5B).

FIG. 7B illustrates an appendage gap 654 between appendages 154 and cartridge bottom 609 into which sidewalls 140 slide when cartridge bottom 609 of removable vaping cartridge 150 is inserted into receptacle portion 155. Airflow within the cartridge is illustrated by the white arrows, and shows that in response to a draw, air is pulled through airflow opening 605 in bottom cover 610, and into heating chamber (or heater chamber) 625, which is so named because the heating chamber 625 is the area in which the pre-vapor formulation is heated to form a vapor. It is in the heating chamber that all or most of the vapor is entrained in the air being drawn through removable vaping cartridge 150. The air including the entrained vapor, sometimes referred to as the inhalable dispersion, is then delivered through vapor delivery channel 219. In some example embodiments, an upper boundary of heating chamber 625 may be defined by and include a bottom surface of porous ceramic body 691, while a lower boundary of heating chamber 625 may, but need not, be defined by a top of airflow passages 607. As shown in FIG. 7B, the top surface of the porous ceramic body 691 includes a recess or indentation for receiving pre-vapor formulation.

In operation, porous ceramic body 691, which is part of heater 203 (illustrated in FIG. 8) absorbs a pre-vapor formulation stored in fluid chamber 221. Power is applied to heating element 803 (illustrated in FIG. 8) via cartridge/pod contacts 601 and contact posts 689, and heating element 803 heats pre-vapor formulation absorbed by porous ceramic body 691 to form the vapor that is entrained in the air as it passes through the heating chamber 625.

Referring next to FIG. 8 heater 203 will be discussed in further detail, accordance with various example embodiments. In various example embodiments, a top portion of heater 203 includes a porous ceramic body 691 is positioned in fluid communication with pre-vapor formulation stored in reservoir or fluid chamber 221 (FIG. 2), and includes an s-shaped conductive heating element 803 inlaid in, or otherwise formed on or attached to, a bottom of the porous ceramic body 691 facing away from reservoir or fluid chamber 221. The shape of heating element 803 is not limited to being S-shaped, but may be another suitable shape configured to contact a desired surface area of porous ceramic body 691.

In the illustrated example embodiment, heater 203 also includes contact posts 689 protruding from, and extending within porous ceramic body 691. Contact posts 689 are electrically connected to heating element 803. The portion of contact posts 689 protruding from ceramic body may be soldered, welded, crimped, or otherwise connected to cartridge/pod contacts 601. In at least one example embodiment, a lower surface 809, which may be the same surface from which contact posts 689 protrude, may define the upper boundary of heating chamber 625. In other example embodiments, heating element 803, which may or may not protrude beyond lower surface 809, may define the upper boundary of heating chamber 625.

In some example embodiments, an e-vaping device may include some or all of the features set forth in U.S. Pat. No. 10,064,432, issued Sep. 4, 2018, the entire contents of which is incorporated herein by reference.

It should be understood that the shape of the battery (or batteries) for the power supply may vary. For example, the battery may be cylindrical, prismatic, disc-shaped, a pouch battery, or any other variation of battery shape known in the art. Additionally, it should be understood that the battery may be any of a variety of types. For example, in one embodiment, the battery may be a rechargeable battery (e.g., lithium-ion). In another embodiment, the battery may be a non-rechargeable battery (e.g., alkaline). In yet another embodiment, the battery may include silver oxide, carbon zinc, cadmium, nickel, or any another material known in the art. Furthermore, the battery may include a primary cell and/or a secondary cell. It will be understood by those of ordinary skill in the art that various changes in form and details of the battery may be made without departing from the spirit and the scope of the invention.

As used herein, a pre-vapor formulation refers to, in various embodiments, a substance (e.g., liquid, wax, gel) that may be transformed into a vapor. For example, the pre-vapor formulation may include, but is not limited to, water, solvents, active ingredients, ethanol, plant extracts, natural or artificial flavors, and/or vapor formers such as glycerin and propylene glycol.

It will be understood that plant extracts may include active ingredients as well as their supporting counterparts (e.g., compounds which assist in the absorption of an active ingredient). Active ingredients may include, but are not limited to, nicotine (tobacco derived nicotine, synthetic nicotine, etc.), caffeine, and/or any number of plant extracts including extracts of medicinal plants. Tobacco derived nicotine may be derived from any member of the genus Nicotiana, including one or more species of tobacco plants, such as Nicotiana rustica and Nicotiana tabacum, and may include a blend of two or more different tobacco varieties. Examples of suitable types of tobacco materials that may be used include, but are not limited to, flue-cured tobacco, Burley tobacco, Dark tobacco, Maryland tobacco, Oriental tobacco, rare tobacco, specialty tobacco, blends thereof, and the like. The extract of a medicinal plant may be a naturally occurring constituent or extract of a medicinal plant that has a medically accepted physiological effect (e.g., therapeutic effect, prophylactic effect). For instance, the medicinal plant may be a cannabis plant or a cannabimimetic plant (i.e., a plant with similar pharmacological effects to those of cannabis). For a cannabis plant, the compound may be a cannabinoid. Cannabinoids interact with receptors in the body to produce a wide range of effects. As a result, cannabinoids have been used for a variety of medicinal purposes (e.g., treatment of pain, nausea, epilepsy, psychiatric disorders). For a cannabimimetic plant, the compound may be a cannabimimetic agent. Cannabimimetic agents interact with receptors in the body to produce similar pharmacological effects as cannabinoids.

Examples of cannabinoids include tetrahydrocannabinolic acid (THCA), tetrahydrocannabinol (THC), cannabidiolic acid (CBDA), cannabidiol (CBD), cannabinol (CBN), cannabicyclol (CBL), cannabichromene (CBC), and cannabigerol (CBG). Tetrahydrocannabinolic acid (THCA) is a precursor of tetrahydrocannabinol (THC), while cannabidiolic acid (CBDA) is precursor of cannabidiol (CBD).

In some example embodiments, in addition to active ingredients, the pre-vapor formulation may include flavorants from natural and/or artificial sources, such as plant extracts (e.g., tobacco extract, cannabis extract, cannabimimetic extract), menthol, mint, and/or vanilla.

It should be understood that when an element or layer is referred to as being “on,” “connected to,” “coupled to,” “attached to,” “adjacent to,” or “covering” another element or layer, it may be directly on, connected to, coupled to, attached to, adjacent to or covering the other element or layer or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or” includes any and all combinations or sub-combinations of one or more of the associated listed items.

It should also be understood that, although the terms first, second, third, etc. may be used herein to describe various elements, elements, regions, layers and/or sections, these elements, elements, regions, layers, and/or sections should not be limited by these terms. These terms are only used to distinguish one element, element, region, layer, or section from another region, layer, or section. Thus, a first element, element, region, layer, or section discussed below could be termed a second element, element, region, layer, or section without departing from the teachings of example embodiments.

Spatially relative terms (e.g., “front,” “back,” “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” when used in this specification, specify the presence of stated features, integers, steps, operations, elements, and/or elements, but do not preclude the presence or addition of one or more other features, integers, steps, operations, elements, elements, and/or groups thereof.

While the term “same” or “identical” is used in description of example embodiments, it should be understood that some imprecisions may exist. Thus, when one element is referred to as being the same as another element, it should be understood that an element or a value is the same as another element within a desired manufacturing or operational tolerance range (e.g., ±10%).

When the terms “about” or “substantially” are used in this specification in connection with a numerical value, it is intended that the associated numerical value includes a manufacturing or operational tolerance (e.g., ±10%) around the stated numerical value. Moreover, when the words “generally” and “substantially” are used in connection with geometric shapes, it is intended that precision of the geometric shape is not required but that latitude for the shape is within the scope of the disclosure. Further, regardless of whether numerical values or shapes are modified as “about” or “substantially,” it will be understood that these values and shapes should be construed as including a manufacturing or operational tolerance (e.g., ±10%) around the stated numerical values or shapes.

Some example embodiments described above include a microcontroller, processor, or the like. A processor, microprocessor, controller, or the like may include processing circuitry such as hardware including logic circuits; a hardware/software combination such as a processor executing software stored in a memory; or a combination thereof. For example, the processing circuitry more specifically may include, but is not limited to, a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), etc. Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

What is claimed is:

1. A receiver assembly of an e-vaping device, the receiver assembly including:
an enclosed portion having an upper surface; and
a receptacle portion configured to receive a cartridge bottom of a removable vaping cartridge, the receptacle portion including
a receptacle bottom defined by the upper surface of the enclosed portion,
front and back flanges having a flange height measured from the receptacle bottom,
sidewalls having a sidewall height measured from the receptacle bottom, and
an upper receptacle boundary defined by the sidewall height, the sidewall height being short enough such that at least a portion of a heating chamber included in a removable vaping cartridge remains above the sidewalls while the cartridge bottom of the removable vaping cartridge is inserted in the receptacle portion.

2. The receiver assembly of claim 1, wherein the flange height is short enough such that the at least a portion of the heating chamber remains above the front and back flanges while the cartridge bottom of the removable vaping cartridge is inserted in the receptacle portion.

3. The receiver assembly of claim 1, wherein the sidewall height is short enough such that a fluid chamber included in the removable vaping cartridge remains above the sidewalls while the cartridge bottom of the removable vaping cartridge is inserted in the receptacle portion.

4. The receiver assembly of claim 3, wherein the flange height is short enough such that the fluid chamber remains above the front and back flanges while the cartridge bottom of the removable vaping cartridge is inserted in the receptacle portion.

5. The receiver assembly of claim 1, wherein the sidewall height is short enough such that a heater included in the removable vaping cartridge remains above the sidewalls while the cartridge bottom of the removable vaping cartridge is inserted in the receptacle portion.

6. The receiver assembly of claim 5, wherein the flange height is short enough such that the heater remains above the front and back flanges while the cartridge bottom of the removable vaping cartridge is inserted in the receptacle portion.

7. The receiver assembly of claim 1, wherein
the sidewall height is short enough such that the at least a portion of the heating chamber, a heater, and a fluid chamber included in the removable vaping cartridge remain above the sidewalls while the cartridge bottom of the removable vaping cartridge is inserted in the receptacle portion.

8. The receiver assembly of claim 1, wherein
the flange height is short enough such that the at least a portion of the heating chamber, a heater, and a fluid chamber included in the removable vaping cartridge remain above the front and back flanges while the cartridge bottom of the removable vaping cartridge is inserted in the receptacle portion.

9. The receiver assembly of claim 1, wherein the front and back flanges include:
ribs configured to engage the cartridge bottom of the removable vaping cartridge.

10. A receiver assembly of an e-vaping device, the receiver assembly including:
an enclosed portion having an upper surface; and
a receptacle portion configured to receive a cartridge bottom of a removable vaping cartridge, the receptacle portion including
a receptacle bottom defined by the upper surface of the enclosed portion,
front and back flanges having a flange height measured from the receptacle bottom,
sidewalls having a sidewall height measured from the receptacle bottom, and
an upper receptacle boundary defined by the sidewall height, the sidewall height being short enough such that a heater included in a removable vaping cartridge remains above the sidewalls while the cartridge bottom of the removable vaping cartridge is inserted in the receptacle portion.

11. The receiver assembly of claim 10, wherein the flange height is short enough such that the heater remains above the front and back flanges while the cartridge bottom of the removable vaping cartridge is inserted in the receptacle portion.

12. The receiver assembly of claim 10, wherein the sidewall height is short enough such that a fluid chamber included in the removable vaping cartridge remains above the sidewalls while the cartridge bottom of the removable vaping cartridge is inserted in the receptacle portion.

13. The receiver assembly of claim 12, wherein the flange height is short enough such that the fluid chamber remains above the front and back flanges while the cartridge bottom of the removable vaping cartridge is inserted in the receptacle portion.

14. The receiver assembly of claim 10, wherein the sidewall height is short enough such that a heating element of the heater remains above the sidewalls while the cartridge bottom of the removable vaping cartridge is inserted in the receptacle portion.

15. The receiver assembly of claim 14, wherein the flange height is short enough such that the heating element of the heater remains above the front and back flanges while the cartridge bottom of the removable vaping cartridge is inserted in the receptacle portion.

16. The receiver assembly of claim 10, wherein
the sidewall height is short enough such that at least a portion of a heating chamber, the heater, a heating element of the heater, and a fluid chamber included in the removable vaping cartridge remain above the sidewalls while the cartridge bottom of the removable vaping cartridge is inserted in the receptacle portion.

17. The receiver assembly of claim 10, wherein the front and back flanges include:
ribs configured to engage the cartridge bottom of the removable vaping cartridge.

18. A receiver assembly of an e-vaping device, the receiver assembly including:
an enclosed portion having an upper surface; and
a receptacle portion configured to receive a cartridge bottom of a removable vaping cartridge, the receptacle portion including
a receptacle bottom defined by the upper surface of the enclosed portion,
front and back flanges having a flange height measured from the receptacle bottom,
sidewalls having a sidewall height measured from the receptacle bottom, and
an upper receptacle boundary defined by the sidewall height, the sidewall height being short enough such that a fluid chamber included in a removable vaping cartridge remains above the sidewalls while the cartridge bottom of the cartridge bottom of the removable vaping cartridge is inserted in the receptacle portion.

19. The receiver assembly of claim 18, wherein the flange height is short enough such that the fluid chamber remains above the front and back flanges while the cartridge bottom of the removable vaping cartridge is inserted in the receptacle portion.

20. The receiver assembly of claim 18, wherein the sidewall height is short enough such that a heater included in the removable vaping cartridge remains above the sidewalls while the cartridge bottom of the removable vaping cartridge is inserted in the receptacle portion.

21. The receiver assembly of claim 20, wherein the flange height is short enough such that the heater remains above the front and back flanges while the cartridge bottom of the removable vaping cartridge is inserted in the receptacle portion.

22. The receiver assembly of claim 20, wherein the sidewall height is short enough such that a heating element of the heater remains above the sidewalls while the cartridge bottom of the removable vaping cartridge is inserted in the receptacle portion.

23. The receiver assembly of claim 20, wherein the front and back flanges include:
ribs configured to engage the cartridge bottom of the removable vaping cartridge.
